(19) United States
(12) Patent Application Publication
(10) Pub. No.: US 2025/0302945 A1
(43) Pub. Date: Oct. 2, 2025

(54) METHOD FOR TREATING GLIOMA WITH OHSV2

(71) Applicant: Binhui Biopharmaceutical Co., Ltd, Wuhan (CN)

(72) Inventor: Binlei LIU, Beijing (CN)

(21) Appl. No.: 18/623,179

(22) Filed: Apr. 1, 2024

(51) Int. Cl. A61K 39/245; A61K 39/00; A61P 35/00

(52) U.S. Cl. CPC A61K 39/245; A61P 35/00; A61K 2039/5256; A61K 2039/54; A61K 2039/545; C12N 2710/16622; C12N 2710/16632; C12N 2710/16662; C12N 2710/16671

(57) ABSTRACT

Provided is a recombinant oncolytic herpes simplex virus type II (oHSV2) and its injection for treating central nervous system (CNS) tumor, such as a recurrent glioma.

### FIELD

The present disclosure relates to the technical field of biological medicine, specifically to a recombinant oncolytic herpes simplex virus type II (oHSV2) and a method for treatment of central nervous system tumors, specifically for treatment of glioma, and more specifically, for treatment of a recurrent glioma, with the oHSV2.

### BACKGROUND

Glioma, accounting for approximately 46% of intracranial tumor cases, is one of the most common primary malignant tumors occurring at the central nervous system. With the highest morbidity among brain tumors, glioma presents a peak of onset age between 30 to 40, or 10 to 20 years old overall. Glioma is of strong aggressiveness, frequent recurrence, and high mortality. At present, the clinical treatment of glioma is mostly based on post-surgical radiotherapy and chemotherapy. However, glioma is resistant to many chemotherapeutic drugs due to different sensitivities against anti-tumor drugs between different individuals, thus resulting in unsatisfactory clinical efficacies.

Glioblastoma multiforme (GBM) is one of the most common malignant primary tumors in brain, the initial standard treatments for which are surgical resection and post-surgical radiotherapy. However, for most patients GBM will recur regardless of which treatment initially adopt, and there are no standard chemotherapy regimens for recurrent glioma after the initial standard treatments. It is still palliative for salvage chemotherapy regimens with single-agent or drug combinations for the recurrent high-grade gliomas of patients, whose median survival is from 6 to 8 months, and very few of them survive more than 2 years. The guidelines on the diagnosis and treatment of gliomas (2022 edition) recommends that for the high-grade recurrent gliomas, clinical trials are strongly advised as a matter of priority, and in the absence of an appropriate clinical trial, the following regimens may be used: a. bevacizumab; b. temozolomide; c. lomustine or carmustine; d. PCV regimen; e. regorafenib; f. bevacizumab plus chemotherapy with carmustine/lomustine or temozolomide; g. etoposide; h. carboplatin or cisplatin-based chemotherapy regimen; i. corresponding target-specific drugs recommendable for patients with BRAF V600E activating mutation or NTRK fusion.

Oncolytic viruses are a class of natural or gene-edited viruses that can specifically replicate in tumor cells and exert anti-tumor effects, and one of them, recombinant oncolytic herpes simplex virus, is an oncolytic virus with development potential for use in cancer immunotherapy. Currently, four oncolytic viral therapies are available around the world, in which adenoviruses, herpesviruses, reoviruses and cowpox viruses are most commonly used, accounting for 31%, 24%, 20% and 12.5% respectively. In June, 2021, oncolytic herpes simplex virus type I, Delytact (G47Δ), provided by Daiichi Sankyo in Japan, was approved for the treatment of malignant glioblastoma. Clinical data showed that the survival rates of malignant glioma patients treated by G47Δ had significantly improved compared with that treated by conventional therapies. However, G47Δ still has the disadvantages of high administration dosage, complex administration means and the need for synchronous administration of other drugs for the treatment.

Therefore, there is an urgent need for providing a new drug and a method for the treatment of patients with brain glioma, by the new drug.

### SUMMARY

Regarding the deficiencies of the related art, in view of the limited therapeutic effects of currently approved drugs for the treatment of glioma, especially malignant glioma, an object of the present disclosure is to provide an antitumor drug with improved treatment efficacy, which is assessed by indicators such as “Objective Response Rate (ORR)”, “Disease Control Rate (DCR)”, “Median Overall Survival (mOS)” and the like, and with fewer side effects for patients suffering from glioma.

Surprisingly, the inventors found that the recombinant oncolytic herpes simplex virus type II (oHSV2) shows an excellent therapeutic effect on glioma in clinical trials, in the form of an injection, indicating that the oHSV2 can effectively treat glioma, especially malignant glioma. More specifically, the oHSV2 also function effectively in subjects intolerant to one or both of chemotherapy and radiotherapy and/or resistant to treatment at least two lines of previous therapy. For the purpose of the present disclosure, the oHSV2, including a stable genomic sequence of a recombinant oncolytic herpes simplex virus type II as described in patent No. CN102146418B, refers to the modified herpes simplex type II virus obtained according to the method therein. As used herein, the term “oHSV2” refers to a recombinant oncolytic herpes simplex virus type II as disclosed in patent No. CN102146418B and Zhao Q, Zhang W, Ning Z, Zhuang X, Lu H, Liang J, Li J, Zhang Y, Dong Y, Zhang Y, Zhang S, Liu S, Liu B. A novel oncolytic herpes simplex virus type 2 has potent anti-tumor activity. PLoS One. 2014 Mar. 26; 9 (3): e93103. doi: 10.1371/journal.pone.0093103. PMID: 24671154; PMCID: PMC3966855. Meanwhile, the oHSV2, as an oncolytic virus capable of killing tumor cells, should be interpreted as an antitumor drug in the particular context of the present disclosure. The entire disclosure of patent No. CN102146418B is incorporated herein by reference.

To realize the above object, the present disclosure provides the following technical solutions.

In a first aspect, the present disclosure, in embodiments, provides an antitumor drug for a central nervous system tumor, such as glioma including brain glioma and the like. The antitumor drug includes recombinant oncolytic herpes simplex virus type II (oHSV2) as active ingredient, which is named as H2d3d4-hGF, with a proposed taxonomic designation of Herpes Simplex Virus Type 2, and is deposited in depository authority of China General Microbiological Culture Collection Center located in Institute of Microbiology, Chinese Academy of Sciences, Building No. 3, Yard No. 1, West Beichen Road, Chaoyang District, Beijing, China, on Feb. 3, 2010, with an accession number of CGMCC No. 3600. Specifically, the oHSV2 is obtained by knocking out genes ICP34.5 and ICP47 of a wild herpes simplex virus type II strain HG52, and inserting a human granulocyte-macrophage colony-stimulating factor (hGM-CSF) cassette at the position of the knocked out gene ICP34.5.

In some embodiments, the antitumor drug is an oHSV2 injection.

In a second aspect, the present disclosure provides a method for treating a subject with a central nervous system tumor, including: administering the subject a therapeutically effective amount of an antitumor drug, where the antitumor drug contains recombinant oncolytic herpes simplex virus type II (oHSV2), which is named as H2d3d4-hGF, with a proposed taxonomic designation of Herpes Simplex Virus Type 2, and is deposited in depository authority of China General Microbiological Culture Collection Center located in Institute of Microbiology, Chinese Academy of Sciences, Building No. 3, Yard No. 1, West Beichen Road, Chaoyang District, Beijing, China, on Feb. 3, 2010, with an accession number of CGMCC No. 3600.

In some embodiments, the central nervous system tumor is a recurrent central nervous system tumor.

In some embodiments, the central nervous system tumor is glioma.

In some embodiments, the glioma is glioblastoma.

In some embodiments, the central nervous system tumor is brain glioma.

In some embodiments, the subject is intolerant to one or both of chemotherapy and radiotherapy.

In some embodiments, the subject is resistant to treatment at least two lines of previous therapy, wherein the at least two lines of previous therapy are selected from first-line, second-line, third-line therapies and immunotherapy beyond line.

In some embodiments, the subject is over 18 years of age.

In some embodiments, for each treatment cycle, the antitumor drug is administered once every 3 weeks, with administration times of ≥3.

In some embodiments, the oHSV2 in the antitumor drug is administered with a single dose from 10⁶ CCID₅₀/ml to 10⁷ CCID₅₀/ml.

In some embodiments, the antitumor drug is administered a single dose or multiple doses.

In some embodiments, the oHSV2 in the antitumor drug is administered with a single dose from 10⁶ CCID₅₀/ml to 10⁷ CCID₅₀/ml with a single administration volume of ≤2 ml.

In some embodiments, the oHSV2 in the antitumor drug is administered with a single dose of 10⁶ CCID₅₀/ml or 10⁷ CCID₅₀/ml with a single administration volume of ≤2 ml.

In some embodiments, the oHSV2 in the antitumor drug is administered with a single dose lower than 2*10⁷ CCID₅₀.

In some embodiments, the antitumor drug is in form of an injection, the method specifically includes: administering the antitumor drug by intratumor injection.

In some embodiments, the oHSV2 in the antitumor drug is formulated in a pharmaceutically acceptable solution.

In some embodiments, the antitumor drug is administered by a direct subcutaneous injection or an ultrasound-guided intratumor injection.

In some embodiments, Ommaya reservoir is used as a device for administering the antitumor drug into the subject.

In some embodiments, the antitumor drug is used in a combination with other antitumor drugs, supportive antitumor drugs and/or drug excipients.

In some embodiments, the oHSV2 is obtained by knocking out genes ICP34.5 and ICP47 in a wild herpes simplex virus type II strain HG52 and inserting a human granulocyte-macrophage colony-stimulating factor (hGM-CSF) cassette at the position of the knocked out gene ICP34.5.

The present disclosure provides an antitumor drug for a central nervous system tumor, such as a brain glioma, and the drug may be an injection containing the oHSV2.

Compared to the related art, the antitumor drug provided by the present disclosure has at least the following beneficial effects.
i) The oHSV2 and its injection provided in the present disclosure are obtained by means of molecular cloning, DNA homologous recombination, and the like, in which the wild herpes simplex virus type II is modified by knocking out neurotoxin and immunosuppressive genes and inserting immune-enhancing factor genes into its viral genome. The knock-out of neurotoxin genes enables the oHSV2 to selectively replicate in tumor cells with impaired PKR signaling pathway and expand to infect the surrounding tumor cells, rather than replicate in normal cells, which results in significantly decreased virulence and reduced drug side effects. The knock-out of the immunosuppressive genes facilitates the activation of anti-tumor immune responses. These two modifications enhance the oncolytic activity of the virus. In addition, insertion of hGM-CSF cassette induces differentiation, proliferation and maturation of tumors and their surrounding dendritic cell (DC) precursors, as well as enhances antigen presenting of DCs to activate immune killer cells in vivo, which contributes to inducing local and systemic anti-tumor immune responses.
ii) The oHSV2 and its injection provided in the present disclosure used for the treatment of recurrent central nervous system tumors show significantly improved ORR, DCR and mOS compared to the related art, achieving more beneficial therapeutic effects and reduced side effects caused by treatment in the meantime.

### BRIEF DESCRIPTION OF THE DRAWINGS

FIG. 1 shows a treatment flow of subject S00103 according to an embodiment of the present disclosure.

FIG. 2 shows a treatment flow of subject S00108 according to an embodiment of the present disclosure.

FIG. 3 shows MRI scan results at brain lesions of subject S00103 before and after the treatment, according to an embodiment of the present disclosure.

FIG. 4 shows MRI scan results at brain lesions of subject S00108 before and after treatment, according to an embodiment of the present disclosure.

FIG. 5 shows a preliminary survival analysis for the treatment of glioma with an oHSV2 injection according to an embodiment of the present disclosure.

### DETAILED DESCRIPTION

In order to make the object, technical solution and advantages of the present disclosure more clearly understood, the present disclosure is further described in detail below with reference to embodiments. It should be understood that the specific embodiments described herein are only intended to explain the present disclosure but not to limit the present disclosure.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

Unless defined otherwise, all scientific and technical terms used herein have the same meaning as is commonly understood by one skilled in the art to which this invention belongs. All patents and publications referred to herein are incorporated by reference in their entirety.

### TERMS

The term “oHSV2 injection” refers to oHSV2 in the form of injection. The term “injection” refers to a sterile solution containing drug and for injecting administration into the body, which includes an emulsion and suspension, as well as sterile powders or a concentrated solution containing drug and for preparation into a solution or suspension before use. The injection works rapidly and reliably and is free from influences of pH, enzymes, food, etc. as well as the first-pass effect, enabling systemic or local effects, and is therefore suitable for patients who are unfit for or cannot perform oral administration of drugs. In the present disclosure, the terms “injection” and “injection preparation” are equivalent in definition.

The terms “Complete Response (CR)”, “Partial Response (PR)”, “Stable Disease (SD)”, “Progressive Disease (PD)” refers to four assessment levels in terms of therapeutic efficacy on tumors (solid tumors only). Specifically, the term “CR” refers to all target lesions have disappeared, no new lesion appears, and a tumor markers are normal, with all of these maintaining for at least 4 weeks; “PR” refers to the sum of the largest diameters of target lesions has been reduced by ≥30%, and maintained for at least 4 weeks; “SD” refers to the sum of the largest diameters of target lesions is reduced but not reaching the criteria of PR, or increased but not reaching the criteria of PD; and “PD” refers to the sum of the largest diameters of target lesions has been increased by at least ≥20%, or new lesions appear.

The terms “Objective Response Rate (ORR)” and “Disease Control Rate (DCR)” refer to associated indicators for assessing the therapeutic efficacy on tumors (solid tumors only). Generally, “ORR” refers to the proportion of subjects whose tumor volumes have reduced by at least 30% and maintained for 4 weeks, that is to say, the sum of the proportions of subjects in CR and PR. The higher the ORR, the more subjects under the treatment having tumor reduced. “DCR” refers to the proportion of subjects that achieve CR+PR and SD under the treatment, i.e., the proportion of subjects who do not experience PD.

The term “Median Overall Survival (mOS)”, also known as half-survival in medicine, refers to the survival time corresponding to a cumulative survival rate of 50%, representing only 50% of the individuals being survival at this point. The mOS is an indicator for assessing the therapeutic efficacy of cancer subject in terms of survival, and is generally used to determine the prognosis of subjects with malignant tumors, where the longer the mOS, the longer overall survival time of subjects with malignant tumors. The mOS may also be used to determine the therapeutic efficacy of a new program. If subjects undergoing a certain treatment program is found to have a prolonged mOS or a significantly improved mOS than that of current standard treatment programs, generally such a new treatment program would be recommended or applied to clinical treatment, so as to bring certain benefits to the subjects such as increasing the survival rate of the subjects and improving the life quality of the subjects.

In embodiments of the present disclosure, the term “preferred” is only used for describing a more effective embodiment or example, and should not constitute a limitation on the protection scope of the present disclosure.

In embodiments of the present disclosure, the technical features described with an open manner both include a technical solution consisting of the enumerated features, and a technical solution including the enumerated features.

In embodiments of the present disclosure, when numerical intervals are involved, unless otherwise specified, endpoints of the numerical interval are included.

In embodiments of the present disclosure, an antitumor drug comprising oHSV2 may in addition comprise at least one pharmaceutically acceptable excipient/solution, e.g. carrier or diluent, e.g. including fillers, binders, disintegrators, flow conditioners, lubricants, sugars and sweeteners, fragrances, preservatives, stabilizers, wetting agents and/or emulsifiers, solubilizers, salts for regulating osmotic pressure and/or buffers.

In embodiments of the present disclosure, first-line, second-line, third-line therapies and immunotherapy beyond line (also named as immunotherapy beyond progression, IBD) may refer to antineoplastic protocols for respective cancers recommended in the related art.

Recombinant oncolytic herpes simplex virus type II (referred to as oHSV2 later) can selectively infect and replicate itself in the tumor cells, ultimately lysing and killing the tumor cells, as well as releasing progeny viral particles to infect the surrounding tumor cells further. This process also contributes to the release of tumor-associated antigens (TAAs).

The anti-tumor effect of the oHSV2 not only lies in directly killing the tumor cells by viral replication or the direct toxicity of viral proteins, but also in the regulation to the immunosuppressive tumor microenvironment, which is conducive to breaking the immune tolerance thereby triggering anti-tumor immune responses, where lysis of the tumor cells results in releases of TAAs, thereby inducing systemic anti-tumor immune responses in the body, according to recent studies.

After extensive and in-depth research, the inventors have applied the oHSV2 and its injection into the treatment of glioma for the first time. Results of the clinical trials proved the effectiveness of the oHSV2 and its injection in the treatment of glioma. On the basis of such research and clinical trials, the present disclosure is provided.

Research on oHSV2 Injection and Anti-Tumor Mechanism Thereof

The recombinant oncolytic herpes simplex virus type II (oHSV2) injection provided in the present disclosure is obtained by subjecting a wild herpes simplex virus type II to modifications of knocking out neurotoxin and immunosuppressive genes and inserting immune-enhancing factor genes into the viral genome, with molecular cloning, DNA homologous recombination, and other techniques. The knock-out of neurotoxin genes enables the oHSV2 to selectively replicate in tumor cells with impaired PKR signaling pathway and expand to infect the surrounding tumor cells, rather than replicate in normal cells, which results in significantly decreased virulence and reduced drug side effects. The knock-out of the immunosuppressive genes facilitates the activation of anti-tumor immune responses. These two modifications enhance the oncolytic activity of the virus. In addition, insertion of hGM-CSF cassette induces differentiation, proliferation and maturation of tumors and their surrounding dendritic cell (DC) precursors, as well as enhances antigen presenting of DC to activate immune killer cells in vivo, which contributes to inducing local and systemic anti-tumor immune responses. It also ensures the oncolytic activity of the oHSV2 and increases its druggability along with immune activation.

The oHSV2 and injection thereof provided in an embodiment of the present disclosure contain a recombinant oncolytic herpes simplex virus type II, which is named as H2d3d4-hGF, with a proposed taxonomic designation of Herpes Simplex Virus Type 2, and is deposited in depository authority of China General Microbiological Culture Collection Center located in Institute of Microbiology, Chinese Academy of Sciences, Building No. 3, Yard No. 1, West Beichen Road, Chaoyang District, Beijing, China, on Feb. 3, 2010, with an accession number of CGMCC No. 3600.

The oHSV2 provided in an embodiment of the present disclosure has been proved to be highly safe and has good therapeutic effects on solid tumors such as colon cancer, liver cancer, lung cancer, melanoma, head and neck tumors and the like based on results of many animal experiments. These experimental studies and results can be used as the research basis of the oHSV2 and injection thereof for use in the treatment of central nervous system tumors, such as glioma including brain glioma and the like, according to embodiments of the present disclosure.

### SPECIFIC EXAMPLE

In a specific example, there is provided a method for treating a central nervous system tumor in a subject comprising administering the subject a therapeutically effective amount of a recombinant oncolytic herpes simplex type II virus, wherein central nervous system tumor is glioma or glioblastoma, and wherein the subject is resistant to treatment at least two lines of previous therapy, and wherein the virus is formulated in a pharmaceutically acceptable solution, and wherein the virus strength is 10⁷ CCID₅₀ viral particles per 1 milliliter of pharmaceutically acceptable solution, and wherein the virus is administered every three weeks, and wherein said triweekly administration consists of a single or multiple direct or ultrasound-guided intratumoral injections, and wherein total viral particles administered per said triweekly injection do not exceed 2*10⁷ CCID₅₀ viral particles, and wherein the patient does not exhibit more than grade III or higher grade, treatment-related toxicities as defined by RECIST criteria within the first 4 administrations of treatment.

Clinical Assessment for Treatment of Glioma with oHSV2 Injection

Examples of the present disclosure provide clinical use of oHSV2 and injection thereof in treatment of CNS tumor. Subjects with melanoma were intracranially administered with effective amounts of oHSV2 multiple times thereby maximizing the therapeutic effectiveness of the oHSV2 and avoiding or reducing side effects resulting from the administration. Specific Examples are described below.

### Example 1. Clinical Trial Design

### 1.1 Phase I Clinical Trial

The primary objective of Phase I clinical trial was to explore the maximum tolerated dose (MTD) and dose limited toxicity (DLT) of the oHSV2 injection by intratumoral administration in subjects with recurrent CNS tumors and have treated with surgical excision, thereby evaluating the safety and tolerability of the injection in the human body. The secondary objective of the phase I clinical trial were as follows: i) to evaluate the preliminary effectiveness of oHSV2 injection in subjects with recurrent CNS tumors treated with intratumorally postoperative administration; ii) to evaluate the biological distribution and shedding of oHSV2 in subjects with recurrent CNS tumors treated with intratumorally postoperative administration; iii) to evaluate the HSV-2 antibody level in subjects with recurrent CNS tumors treated with intratumorally postoperative administration of oHSV2 injection; iv) to determine Recommended Phase II Dose (RP2D) of oHSV2 injection to subjects with recurrent CNS tumors treated with intratumorally postoperative administration, and the like.

Phase I enrolled subjects were patients with CNS tumors treated surgically after clinical recurrence. In Phase I clinical trial, the dose escalation study of oHSV2 injection was tested with two groups, a high dose group (10⁷CCID₅₀/ml) and a low dose group (10⁶ CCID₅₀/ml) via OMMAYA reservoir for injection administration. The total volume of each dose group did not exceed 2 ml per administration according to the size of tumor cavity. The administration was performed once every 3 weeks, and the total administration number was from 3 to 6 times.

### 1.2 Phase IIa Clinical Trial

The primary objective of Phase IIa clinical trial was to evaluate the preliminary effectiveness of oHSV2 injection in subjects with recurrent glioblastoma treated with intratumorally postoperative administration; and the secondary objective of Phase IIa clinical trial was to further evaluate the safety of oHSV2 injection for the treatment of recurrent glioblastoma.

The subjects enrolled in phase IIa clinical trial were patients with recurrent glioblastoma and have treated with surgical excision after the recurrence, and the administration route, frequency and number were the same as that in phase I, where the dosage was in line with RP2D determined in phase I.

### 2. Criteria for Subject Enrollment

### 2.1 Main Enrollment Criteria

(1) Subjects, whose age was ≥18, were selected, with gender unlimited;
(2) For phase I clinical trial, patients with pathologically confirmed recurrent central nervous system tumors and had been treated with surgical operation after recurrence, were selected;
(3) For phase IIa clinical trial, patients with pathologically confirmed recurrent glioblastoma and had been treated with surgical operation after recurrence, were selected;
(4) Subjects, whose KPS score ≥60, were selected;
(5) Subjects, for which partial or complete tumor resection were achieved via surgery and OMMAYA reservoir had been placed in the surgical area, or substantial resection had not been operated, but OMMAYA reservoir had been placed in the surgical area and met the requirement of administration, were selected.

### 2.2 Main Exclusion Criteria

(1) Subjects, who had received tumor chemotherapy, targeted therapy, or immunotherapy within 28 days before the first administration of the tested drug, were excluded;
(2) Subjects, who had received antiviral medication within 28 days before the first administration of the tested drug, were excluded;
(3) Subjects, who had received radiation therapy to the brain within 3 months before the first administration of the tested drug, were excluded;
(4) Subjects, with other active extracranial malignant tumors requiring concurrent treatment, were excluded.

### 3. Enrolled Subjects

A total of 15 subjects were enrolled in the Example, and their baseline characteristics were shown in Table 1 below.

TABLE 1

| | | | | | | Past | |
|---|---|---|---|---|---|---|---|
| | | | Disease | KPS | | Treatment | Enrolled |
| No. | Sex | Age | diagnosis | score | Past treatment history | Outcome | Group |
| S00101 | female | 53 | Glioblastoma | 80 | Radiotherapy, original tumor | Progression, | Low dose |
| | | | | | resection of right temporal- | recurrence | group |
| | | | | | occipital craniotomy, | | |
| | | | | | craniotomy treatment, tumor | | |
| | | | | | resection of right temporal- | | |
| | | | | | occipital craniotomy via the | | |
| | | | | | original incision + artificial | | |
| | | | | | dural repair. | | |
| S00102 | Male | 31 | Glioblastoma | 60 | Tumor resection at skull base | Progression, | Low-dose |
| | | | | | (complicated), tumor | recurrence | group |
| | | | | | resection at skull base | | |
| | | | | | (complicated). | | |
| S00103 | Male | 23 | Glioblastoma | 60 | Radiotherapy, TMZ, | Progression, | Low dose |
| | | | | | nedaplatin, TG02 capsule, | recurrence | group |
| | | | | | dianhydrogalactitol, apatinib, | | |
| | | | | | PD1, craniotomy for | | |
| | | | | | resection of right temporal | | |
| | | | | | glioma, tumor resection | | |
| | | | | | through right frontal | | |
| | | | | | temporal zygomatic arch | | |
| | | | | | approach (Dolenc approach) | | |
| | | | | | via original incision, | | |
| | | | | | microresection of recurrent | | |
| | | | | | glioma in right | | |
| | | | | | frontotemporal insula with | | |
| | | | | | the assistance of yellow | | |
| | | | | | fluorescence and | | |
| | | | | | electrophysiology + artificial | | |
| | | | | | dural repair. | | |
| S00104 | Male | 40 | Glioblastoma | 70 | Temozolomide, | Progression, | Low dose |
| | | | | | bevacizumab, radiotherapy, | recurrence | group |
| | | | | | microscopic resection of | | |
| | | | | | deep supratentorial lesion + | | |
| | | | | | repair of cerebrospinal fluid | | |
| | | | | | fistula, microscopic resection | | |
| | | | | | of deep supratentorial tumor + | | |
| | | | | | artificial dural repair + | | |
| | | | | | OMMAYA reservoir | | |
| | | | | | implantation, microscopic | | |
| | | | | | resection of deep | | |
| | | | | | supratentorial lesion + | | |
| | | | | | cerebrospinal fluid fistula | | |
| | | | | | repair. | | |
| S00106 | Male | 42 | Glioblastoma | 80 | Radiotherapy, temozolomide, | Progression, | Low dose |
| | | | | | electric field therapy, | recurrence | group |
| | | | | | craniotomy for intracranial | | |
| | | | | | lesion resection through left | | |
| | | | | | frontal, left frontal-temporal- | | |
| | | | | | insula-basal ganglia region | | |
| | | | | | space-occupying lesions + | | |
| | | | | | bone flap reposition and | | |
| | | | | | fixation. | | |
| S00107 | Male | 31 | Glioblastoma | 60 | Radiotherapy, chemotherapy, | Progression, | Low dose |
| | | | | | right temporoparietal brain | recurrence | group |
| | | | | | tumor resection, right | | |
| | | | | | temporal craniotomy for | | |
| | | | | | intracranial resection of | | |
| | | | | | space-occupying lesions via | | |
| | | | | | original incision, right | | |
| | | | | | parietal occipital craniotomy | | |
| | | | | | for tumor resection via | | |
| | | | | | original incision + artificial | | |
| | | | | | dural repair. | | |
| S00108 | Female | 38 | Anaplastic | / | Radiotherapy, chemotherapy, | Progression, | Low-dose |
| | | | astrocytoma | | insula glioma resection | recurrence | group |
| | | | | | through modifed classic left | | |
| | | | | | pterional and Sylvian fissure | | |
| | | | | | approach, left temporal | | |
| | | | | | craniotomy for intracranial | | |
| | | | | | tumor resection. | | |
| S00109 | Male | 54 | Glioblastoma | 60 | Radiotherapy, temozolomide, | Progression, | Low dose |
| | | | | | temozolomide + cisplatin, | recurrence | group |
| | | | | | right parietal occipital | | |
| | | | | | craniotomy for tumor | | |
| | | | | | resection under general | | |
| | | | | | anesthesia. | | |
| S00110 | Male | 55 | Glioblastoma | 60 | Temozolomide, electric field | Progression, | High dose |
| | | | | | therapy, proton therapy, | recurrence | group |
| | | | | | bevacizumab, resection of | | |
| | | | | | temporal lobe lesions, left | | |
| | | | | | temporal parietal craniotomy | | |
| | | | | | for tumor resection via | | |
| | | | | | original incision, OMMAYA | | |
| | | | | | reservoir implantation in the | | |
| | | | | | tumor cavity. | | |
| S00111 | Female | 35 | Oligoastrocytoma | 60 | recurrence after surgery + | Progression, | High dose |
| | | | | | radio-chemotherapy + | recurrence | group |
| | | | | | resection of space-occupying | | |
| | | | | | lesions through right | | |
| | | | | | frontotemporal approach, | | |
| | | | | | stereotactic biopsy (robot- | | |
| | | | | | assisted), resection of skull | | |
| | | | | | base tumor, OMMAYA | | |
| | | | | | reservoir implantation in | | |
| | | | | | tumor cavity | | |
| S00112 | male | 52 | Glioblastoma | 70 | recurrence after surgery + | Progression, | High dose |
| | | | | | radio-chemotherapy, | recurrence | group |
| | | | | | resection of frontal lobe | | |
| | | | | | lesions+ OMMAYA reservoir | | |
| | | | | | implantation in tumor cavity | | |
| S00113 | female | 53 | Diffuse | 70 | recurrence after surgery + | Progression, | High dose |
| | | | astrocytoma | | radio-chemotherapy + | recurrence | group |
| | | | | | resection of fronto-temporal- | | |
| | | | | | insula lesion, resection of | | |
| | | | | | corpus callosum lesion, left | | |
| | | | | | craniotomy for tumor | | |
| | | | | | resection, OMMAYA | | |
| | | | | | reservoir implantation in | | |
| | | | | | tumor cavity | | |
| S00114 | female | 67 | Anaplastic | 70 | recurrence after surgery + | Progression, | High dose |
| | | | pleomorphic- | | radio-chemotherapy, | recurrence | group |
| | | | xanthoastrocytoma | | OMMAYA reservoir | | |
| | | | | | implantation in tumor cavity | | |
| S00115 | female | 58 | Glioblastoma | 70 | recurrence after surgery + | Progression, | High dose |
| | | | (right frontal | | radio-chemotherapy, | recurrence | group |
| | | | lobe) | | OMMAYA reservoir | | |
| | | | | | implantation in tumor cavity | | |
| S00116 | female | 65 | Glioblastoma | 70 | recurrence after surgery + | Progression, | High-dose |
| | | | | | chemotherapy, OMMAYA | recurrence | group |
| | | | | | reservoir implantation in | | |
| | | | | | tumor cavity | | |

### 3. Results of Clinical Trials

### 3.1 Safety

The occurrences of adverse events were shown in Table 2.

TABLE 2

| | 10⁶ CCID50/mL | | | | | | | | 10⁷ CCID50/mL | | | | | | | |
|---|---|---|---|---|---|---|---|---|---|---|---|---|---|---|---|---|
| | N = 5 | | | | | | | | N = 5 | | | | | | | |
| | Grade 1/2 | | | | Grade 3/4 | | | | Grade 1/2 | | | | Grade 3/4 | | | |
| | Number of | | | | Number of | | | | Number of | | | | Number of | | | |
| Classification | subjects | | | | subjects | | | | subjects | | | | subjects | | | |
| of system organs | involved | | | | involved | | | | involved | | | | involved | | | |
| Preferred | in cases | | Times | | in cases | | Times | | in cases | | Times | | in cases | | Times | |
| terminology | (%) | | of cases | | (%) | | of cases | | (%) | | of cases | | (%) | | of cases | |
| Total | 4 | (100.0) | 30 | (100.0) | 3 | (100.0) | 4 | (100.0) | 4 | (100.0) | 16 | (100.0) | 2 | (100.0) | 3 | (100.0) |
| Metabolic and | 1 | (25.0) | 1 | (3.3) | | | | | | | | | | | | |
| Nutritional | | | | | | | | | | | | | | | | |
| Diseases | | | | | | | | | | | | | | | | |
| Hypokalemia | 1 | (25.0) | 1 | (3.3) | | | | | | | | | | | | |
| Infectious and | 1 | (25.0) | 1 | (3.3) | | | | | | | | | | | | |
| invasive | | | | | | | | | | | | | | | | |
| diseases | | | | | | | | | | | | | | | | |
| Infectious fever | 1 | (25.0) | 1 | (3.3) | | | | | | | | | | | | |
| Various types | 2 | (50.0) | 2 | (6.6) | | | | | 1 | (20.0) | 1 | (6.3) | | | | |
| of examination | | | | | | | | | | | | | | | | |
| Elevated blood | 1 | (25.0) | 1 | (3.3) | | | | | | | | | | | | |
| triglycerides | | | | | | | | | | | | | | | | |
| Elevated blood | 1 | (25.0) | 1 | (3.3) | | | | | | | | | | | | |
| homocysteine | | | | | | | | | | | | | | | | |
| Elevated blood | | | | | | | | | 1 | (20.0) | 1 | (6.3) | | | | |
| bilirubin | | | | | | | | | | | | | | | | |
| Various | 3 | (75.0) | 10 | (30.0) | 2 | (66.7) | 3 | (75.0) | 3 | (75.0) | 5 | (31.3) | 2 | (100.0) | 2 | (66.7) |
| neurological | | | | | | | | | | | | | | | | |
| disorders | | | | | | | | | | | | | | | | |
| Headache | 3 | (75.0) | 8 | (26.7) | | | | | 3 | (75.0) | 4 | (25.0) | | | | |
| Cerebral edema | 1 | (25.0) | 1 | (3.3) | 2 | (66.7) | 2 | (50.0) | | | | | 2 | (100.0) | 2 | (66.7) |
| Neuralgia | | | | | 1 | (33.3) | 1 | (25.0) | | | | | | | | |
| Hydrocephalus | 1 | (25.0) | 1 | (3.3) | | | | | | | | | | | | |
| Epilepsy | | | | | | | | | 1 | (20.0) | 1 | (6.3) | | | | |
| Various types | | | | | 1 | (33.3) | 1 | (25.0) | | | | | 1 | (50.0) | 1 | (33.3) |
| of injuries, | | | | | | | | | | | | | | | | |
| poisoning and | | | | | | | | | | | | | | | | |
| complications | | | | | | | | | | | | | | | | |
| of operation | | | | | | | | | | | | | | | | |
| Cerebral hernia | | | | | 1 | (33.3) | 1 | (25.0) | | | | | 1 | (50.0) | 1 | (33.3) |
| Various | 1 | (25.0) | 1 | (3.3) | | | | | | | | | | | | |
| musculoskeletal | | | | | | | | | | | | | | | | |
| and connective | | | | | | | | | | | | | | | | |
| tissue diseases | | | | | | | | | | | | | | | | |
| Myalgia | 1 | (25.0) | 1 | (3.3) | | | | | | | | | | | | |
| Respiratory, | 1 | (25.0) | 1 | (3.3) | | | | | | | | | | | | |
| thoracic and | | | | | | | | | | | | | | | | |
| mediastinal | | | | | | | | | | | | | | | | |
| diseases | | | | | | | | | | | | | | | | |
| Hiccup | 1 | (25.0) | 1 | (3.3) | | | | | | | | | | | | |
| Systemic | 3 | (75.0) | 11 | (36.7) | | | | | 3 | (75.0) | 8 | (50.0) | | | | |
| diseases and | | | | | | | | | | | | | | | | |
| various | | | | | | | | | | | | | | | | |
| reactions at the | | | | | | | | | | | | | | | | |
| administration | | | | | | | | | | | | | | | | |
| site | | | | | | | | | | | | | | | | |
| Fever | 2 | (50.0) | 7 | (23.3) | | | | | 3 | (75.0) | 8 | (50.0) | | | | |
| Influenza-like | 1 | (25.0) | 3 | (10.0) | | | | | | | | | | | | |
| illness | | | | | | | | | | | | | | | | |
| Weakness | 1 | (25.0) | 1 | (3.3) | | | | | | | | | | | | |
| Gastrointestinal | 1 | (25.0) | 3 | (10.0) | | | | | 1 | (20.0) | 2 | (12.5) | | | | |
| system diseases | | | | | | | | | | | | | | | | |
| Nausea | 1 | (25.0) | 2 | (6.6) | | | | | 1 | (20.0) | 2 | (12.5) | | | | |
| Vomiting | 1 | (25.0) | 1 | (3.3) | | | | | | | | | | | | |

As shown in Table 2, a total of 53 times of adverse events (AEs) occurred in 10 subjects out of 15 subjects, most of the AEs had severity at grade 1/2 with common events of headache, fever, and influenza-like illness thereamong. A total of 4 times of serious AEs occurred in 3 subjects, specifically including cerebral edema (grade 3, 2 subjects and 2 times) and cerebral herniation (grade 4, 2 subjects and 2 times), which potentially related to the tested drug. No dose-limiting toxicity (DLT) occurred. The above results indicated that oHSV2 injection and a treatment device thereof were safe in the treatment of glioma.

### 3.2 Effectiveness Assessments

### 3.2.1 Treatment Process and Effectiveness Assessment on Individual Subject

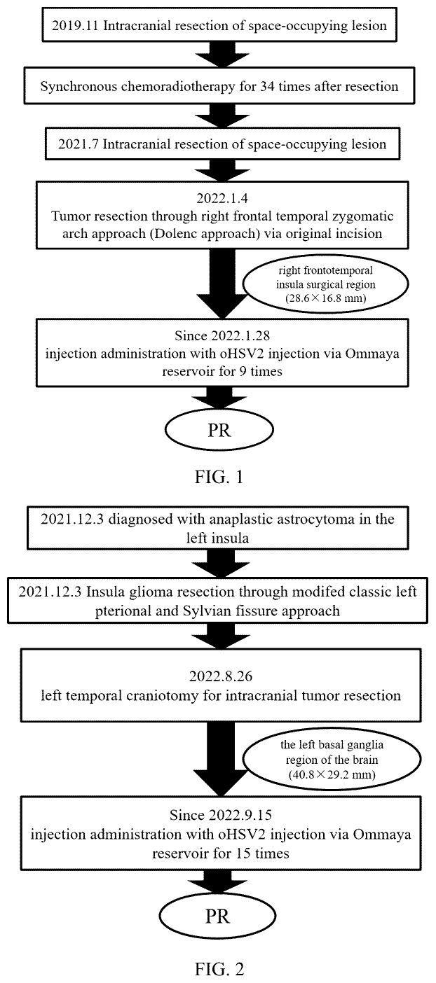

Treatment stages of subject S00103 above were described in further details. The treatment process of subject S00103 was shown in FIG. 1, in which the OMMAYA reservoir was placed at the right frontotemporal insula surgical region (28.6×16.8 mm). The oHSV2 injection was administered to the subject via the OMMAYA reservoir at the low dose (10⁶ CCID₅₀/ml) every three weeks for a total of nine times, via injection.

Treatment stages of subject S00108 above were described in further details. The treatment process of subject S00108 was shown in FIG. 2, in which the OMMAYA reservoir was placed at the left basal ganglia region of the brain (40.8×29.2 mm). The oHSV2 injection was administered to the subject via the OMMAYA reservoir at the low dose (10⁶ CCID₅₀/ml) every three weeks for a total of fifteen times, via injection.

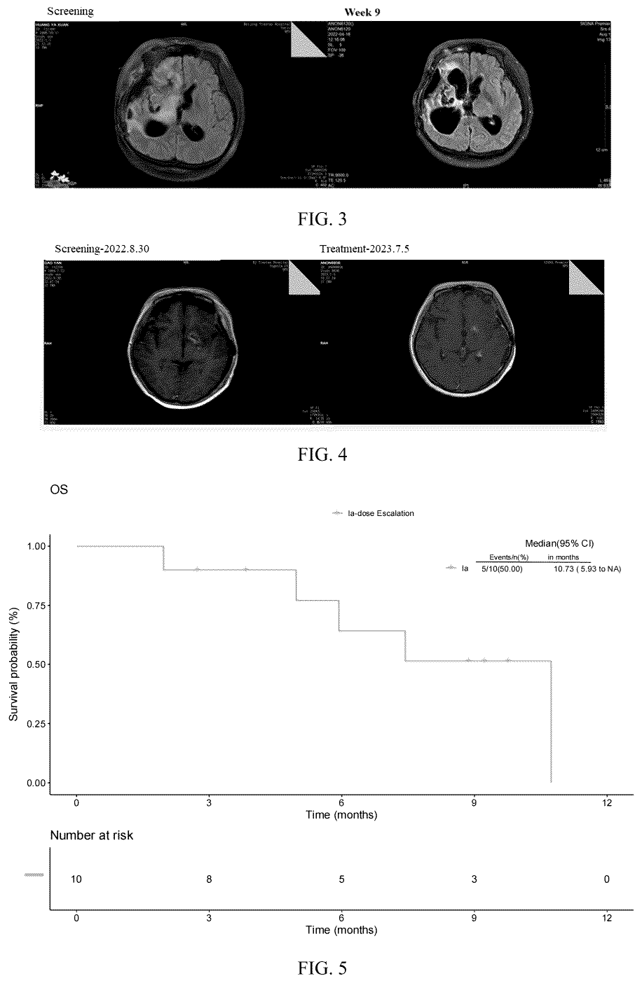

In this Example of the present disclosure, MRI scan at the lesion was performed on subject S00103, who had been treated with oHSV2 injection via OMMAYA reservoir administration. As shown in FIG. 3, the sum of maximum cross-sectional area of the residual lesions of the postoperative subject was 1,191.36 mm³ when enrolled. After 3 times of oHSV2 injections, the sum of maximum cross-sectional area of the residual lesions was reduced to 387.44 mm³, which was a 67.5% reduction compared with baseline, and effectiveness was assessed as PR. MRI scan at the lesion was performed on subject S00108, who had been treated with oHSV2 injection via OMMAYA reservoir administration. As shown in FIG. 4, the sum of maximum cross-sectional area of the residual lesions of the postoperative subject was 480.48 mm³ when enrolled. After 13 times of oHSV2 injections, the sum of maximum cross-sectional area of the residual lesions was reduced to 219.96 mm³, which was a 54.2% reduction compared with baseline, and effectiveness was assessed as PR.

In this Example, blood, and cerebrospinal fluid samples of subject S00103 were collected at different times before and after the administration of oHSV2 injection to detect DNA copies of oHSV2 in the samples, and the detection results are presented in Table 3. As shown in Table 3, at 24 hours and 21 days after dosing, DNA copies of oHSV2 were detected in the cerebrospinal fluid samples of subject S00103, with levels of 15.52 copies/μl and 169.92 copies/μl, respectively, indicating that oHSV2 in form of an injection successfully infected the tumor cells, and replicated and proliferated in the tumor cells, and released its progeny viruses into the cerebrospinal fluid.

TABLE 3

| | | Detected OH2 copy number |
|---|---|---|
| Sample types | Collection time | (copies/μl) |
| Cerebrospinal | D 1 24 h | 15.519 |
| fluid | D 21 24 h (24 h before | 169.922 |
| | the 2nd dosing) | |

### 3.2.2 Assessment of Overall Efficacy

The results of oHSV2 injection for the treatment of glioma are shown in Table 4.

TABLE 4

| Enrollment | | | | | Overall efficacy assessment (iRANO standard) | | | | |
|---|---|---|---|---|---|---|---|---|---|
| Program | | | Ia/Ib | Number of | Assessed | Unassessed | | | |
| number | Cohort | Total | Enrollment | subjects | patients | patients | PR | SD/iSD | iuPD |
| BH-OH2-015 | BH-OH2-015 | 15 | Phase I- | 8 | 5 | 3 | 2 | 1 | 2 |
| | | | 10⁶CCID₅₀/ml | | | | | | |
| | | | Phase I- | 7 | 2 | 5 | 0 | 0 | 2 |
| | | | 10⁷CCID₅₀/ml | | | | | | |

As shown in Table 4, among the 15 enrolled subjects in total, 8 cases of which, enrolled into the low dose group, were treated with the low dose (10⁶ CCID₅₀/ml), while 7 cases of which, enrolled into the high dose group, were treated with high dose (10⁷ CCID₅₀/ml). 7 cases out of the 15 enrolled subjects were assessed, and the other 8 cases did not reached the designed assessment time. According to the iRANO efficacy assessment standard, the treatment results were as follows: 2 patients were assessed as PR, 1 patient was assessed as SD, and 4 patients were assessed as PD, with ORR for 28.6% and DCR for 42.9%, demonstrating a well efficacy of the oHSV2 injection in the treatment of glioma.

In this Example, DNA copies of the oHSV2 were not detected in blood, saliva, and urine samples of all subjects. Further, no live oHSV2 was detected in the shed skin samples derived from the injection site and conjunctival samples, which were collected by wiping. Therefore, the treatment with oHSV2 injection via OMMAYA reservoir administration did not cause any shedding of oHSV2.

3.2.3 Survival Data in Glioma Treatment with oHSV2 Injection

FIG. 5 shows the preliminary survival analysis for the treatment of glioma with the oHSV2 injection. It can be found that the median Overall Survival (mOS) of 10 cases was 10.73 months, the other 5 enrolled cases were not included in the survival analysis set because their survival period were too short.

3.2.4 Comparison of Therapeutic Effects Between oHSV2 Injection and Common Glioma Drugs

The comparison data showing the effectiveness of the oHSV2 injection and a common oncolytic virus drug G47Δ on the treatment of glioma are shown in Table 5 below.

TABLE 5

| | G47Δ | OH2 |
|---|---|---|
| HSV Type | Type I | Type II |
| Enrollment criteria | Patients after primary surgery | Patients after secondary surgery |
| Tumor load at baseline | Small (less than 7 cm²) | Large (average of 27 cm²) |
| Administrated dose | 10⁹ CCID₅₀/ml, ≤6 times | 10⁶ CCID₅₀/ml or 10⁷ |
| | with 4-week intervals | CCID₅₀/ml, ≥3 times |
| Administration route | Positioning plus catheter | Preferred OMMAYA |
| | | reservoir administration |
| ORR | 1/19 | 2/7 with additional 1/7 SD |
| Medicament for treating | Bevacizumab | None |
| recurrence after virotherapy | | |

As shown in Table 5, with the treatments of oHSV2 injection+OMMAYA reservoir administration at the low dose of 10⁶ CCID₅₀/ml and high dose of 10⁷CCID₅₀/ml, these treatments showed a good efficacy on subjects with brain glioma, even in low dose group. By contrast, other products on the market, such as G47Δ, were administered at a dose of 10⁹ CCID₅₀/ml. This result further indicated that the dosage in clinical use of the oHSV2 injection provided by the present disclosure in the treatment of glioma was significantly lower than that of other drugs, thereby further lowering the cost and side effects of drugs treating glioma.

In summary, in the treatment of patients with brain glioma, the oHSV2 injection provided by Examples of the present disclosure can be repeatedly administered without re-operation and has a lower cost and better efficacy than the prior products on the market.

What is described above is only a better specific embodiment of the present disclosure, the scope of protection of the present disclosure is not limited thereto. Any changes or substitutions that can be readily thought of by any person skilled in the art within the technical scope disclosed in the present disclosure shall be covered by the scope of protection of the present disclosure.

What is claimed is:

1. A method for treating a subject with a central nervous system tumor, comprising:
administering the subject a therapeutically effective amount of an antitumor drug,
wherein the antitumor drug comprises recombinant oncolytic herpes simplex virus type II (oHSV2) with a deposit number of CGMCC No. 3600 as an active ingredient.

2. The method according to claim 1, wherein the central nervous system tumor is a recurrent central nervous system tumor.

3. The method according to claim 2, wherein the central nervous system tumor is glioma.

4. The method according to claim 3, wherein the glioma is glioblastoma.

5. The method according to claim 2, wherein the central nervous system tumor is brain glioma.

6. The method according to claim 1, wherein the subject is intolerant to one or both of chemotherapy and radiotherapy.

7. The method according to claim 1, wherein the subject is resistant to treatment at least two lines of previous therapy, wherein the at least two lines of previous therapy are selected from first-line, second-line, third-line therapies and immunotherapy beyond line.

8. The method according to claim 1, wherein the subject is over 18 years of age.

9. The method according to claim 1, wherein for each treatment cycle, the antitumor drug is administered once every 3 weeks, with administration times of ≥3.

10. The method according to claim 1, wherein the oHSV2 in the antitumor drug is administered with a single dose from 10⁶ CCID₅₀/ml to 10⁷ CCID₅₀/ml.

11. The method according to claim 10, wherein the antitumor drug is administered as a single dose or multiple doses.

12. The method according to claim 1, wherein the oHSV2 in the antitumor drug is administered with a single dose from 10⁶ CCID₅₀/ml to 10⁷ CCID₅₀/ml with a single administration volume of ≤2 ml.

13. The method according to claim 1, wherein the oHSV2 in the antitumor drug is administered with a single dose of 10⁶ CCID₅₀/ml or 10⁷ CCID₅₀/ml with a single administration volume of ≤2 ml.

14. The method according to claim 1, wherein the oHSV2 in the antitumor drug is administered with a single dose lower than 2*10⁷ CCID₅₀.

15. The method according to claim 1, wherein the antitumor drug is in form of an injection, the method specifically comprises:
administering the antitumor drug by intratumor injection.

16. The method according to claim 15, wherein the oHSV2 in the antitumor drug is formulated in a pharmaceutically acceptable solution.

17. The method according to claim 1, wherein the antitumor drug is administered by a direct subcutaneous injection or an ultrasound-guided intratumor injection.

18. The method according to claim 1, wherein Ommaya reservoir is used as a device for administering the antitumor drug into the subject.

19. The method according to claim 1, wherein the antitumor drug is used in a combination with supportive antitumor drugs or drug excipients.

20. The method according to claim 1, wherein the oHSV2 is obtained by knocking out genes ICP34.5 and ICP47 in a wild herpes simplex virus type II strain HG52 and inserting a human granulocyte-macrophage colony-stimulating factor (hGM-CSF) cassette at the position of the knocked out gene ICP34.5.
